SEQUENCE LISTING (91 sequences)

SEQ ID NO: 1 - protein, 17 aa, synthetic construct
AAETAVGVLSQFGQEHR

SEQ ID NO: 2 - protein, 13 aa, synthetic construct
ACFYVELIRGRPK

SEQ ID NO: 3 - protein, 14 aa, synthetic construct
ACQKSLRPVPPSPK

SEQ ID NO: 4 - protein, 17 aa, synthetic construct
WESLSTFAASLLAIKPR

SEQ ID NO: 5 - protein, 14 aa, synthetic construct
YGLKGGPSTEGVPK

SEQ ID NO: 6 - protein, 12 aa, synthetic construct
YGYIIEEYGKGR

SEQ ID NO: 7 - protein, 12 aa, synthetic construct
YLTDMTLEEMSR

SEQ ID NO: 8 - protein, 20 aa, synthetic construct
YVLFHTSLLNESNASMGKYK

SEQ ID NO: 9 - protein, 12 aa, synthetic construct
YVTDMTPEEMSR

SEQ ID NO: 10 - protein, 14 aa, synthetic construct
AAEIRASANLAATK

SEQ ID NO: 11 - protein, 13 aa, synthetic construct
AAGDFLGVESMMK

SEQ ID NO: 12 - protein, 16 aa, synthetic construct
ADEICIGYLSNNSTDK

SEQ ID NO: 13 - protein, 12 aa, synthetic construct
YGYIIEEYGKGR

SEQ ID NO: 14 - protein, 14 aa, synthetic construct
YMDLKVEGTTCFYR

SEQ ID NO: 15 - protein, 12 aa, synthetic construct
YVTDMTPEEMSR

SEQ ID NO: 16 - protein, 17 aa, synthetic construct
AAETAVGVLSQFGQEHR

SEQ ID NO: 17 - protein, 17 aa, synthetic construct
AAIIQMENNPDETVCNR

SEQ ID NO: 18 - protein, 14 aa, synthetic construct
ACQKSLRPVPPSPK

SEQ ID NO: 19 - protein, 12 aa, synthetic construct
VFYEGVNFSPRK

SEQ ID NO: 20 - protein, 19 aa, synthetic construct
VLRENAIDTGDGCFEILHK

SEQ ID NO: 21 - protein, 20 aa, synthetic construct
YICDYLEMKPGVYNATTFGK

SEQ ID NO: 22 - protein, 17 aa, synthetic construct
AAETAVGVLSQFGQEHR

SEQ ID NO: 23 - protein, 16 aa, synthetic construct
ADEICIGYLSNNSTDK

SEQ ID NO: 24 - protein, 12 aa, synthetic construct
ADYTLDEESRAR

SEQ ID NO: 25 - protein, 17 aa, synthetic construct
TTPRPLRLPDGPLCSQR

SEQ ID NO: 26 - protein, 13 aa, synthetic construct
VGGNYNYLYRLFR

SEQ ID NO: 27 - protein, 12 aa, synthetic construct
YTVFEAGHTTVR

SEQ ID NO: 28 - protein, 12 aa, synthetic construct
AATHVGKQIVEK

SEQ ID NO: 29 - protein, 16 aa, synthetic construct
ADEICIGYLSNNSTDK

SEQ ID NO: 30 - protein, 12 aa, synthetic construct
ADYTLDEESRAR

SEQ ID NO: 31 - protein, 20 aa, synthetic construct
VNTLTERGVEVVNATETVER

SEQ ID NO: 32 - protein, 14 aa, synthetic construct
VQAHEDEPLQTLYK

SEQ ID NO: 33 - protein, 18 aa, synthetic construct
YVLFHTSLLNESNASMGK

SEQ ID NO: 34 - protein, 17 aa, synthetic construct
AAETAVGVLSQFGQEHR

SEQ ID NO: 35 - protein, 15 aa, synthetic construct
AAGDFLGVESMMKMR

SEQ ID NO: 36 - protein, 12 aa, synthetic construct
ADDSYFQSKFGK

SEQ ID NO: 37 - protein, 12 aa, synthetic construct
TKVPVMGIDSIK

SEQ ID NO: 38 - protein, 12 aa, synthetic construct
WLIAECRNILTK

SEQ ID NO: 39 - protein, 19 aa, synthetic construct
WLLSDTDNAAFPQMTKSYK

SEQ ID NO: 40 - protein, 12 aa, synthetic construct
YLTDMTLEEMSR

SEQ ID NO: 41 - protein, 13 aa, synthetic construct
YSFFFNDEKDGWK

SEQ ID NO: 42 - protein, 15 aa, synthetic construct
YSTLRDQTDPILIMK

SEQ ID NO: 43 - protein, 6 aa, synthetic construct
FEIFPK

SEQ ID NO: 44 - protein, 10 aa, synthetic construct
TLDFHDSNVK

SEQ ID NO: 45 - protein, 13 aa, synthetic construct
NVTVTHAQDILEK

SEQ ID NO: 46 - protein, 8 aa, synthetic construct
NVTVTHAK

SEQ ID NO: 47 - protein, 14 aa, synthetic construct
ADVSAFLRMNLFQK

SEQ ID NO: 48 - protein, 13 aa, synthetic construct
AMMKLTNSAEVAK

SEQ ID NO: 49 - protein, 14 aa, synthetic construct
ASKYFIVGLSCLYK

SEQ ID NO: 50 - protein, 12 aa, synthetic construct
WQRQPQDGYLYR

SEQ ID NO: 51 - protein, 12 aa, synthetic construct
WVKQVVHMLSQR

SEQ ID NO: 52 - protein, 12 aa, synthetic construct
WVQEYINYLELK

SEQ ID NO: 53 - protein, 18 aa, synthetic construct
YIKPQSQFIVDLGAYLEK

SEQ ID NO: 54 - protein, 14 aa, synthetic construct
YVDSEGHLYTVPIR

SEQ ID NO: 55 - protein, 13 aa, synthetic construct
AENGPGPGVLVLR

SEQ ID NO: 56 - protein, 13 aa, synthetic construct
AFCQSPSLIKHQR

SEQ ID NO: 57 - protein, 20 aa, synthetic construct
AMLSGPGQFAENETNEVNFR

SEQ ID NO: 58 - protein, 12 aa, synthetic construct
VTLTTVTVQTIK

SEQ ID NO: 59 - protein, 12 aa, synthetic construct
WVKQVVHMLSQR

SEQ ID NO: 60 - protein, 18 aa, synthetic construct
YIKPQSQFIVDLGAYLEK

SEQ ID NO: 61 - protein, 13 aa, synthetic construct
YINWNFIRICNDR

SEQ ID NO: 62 - protein, 14 aa, synthetic construct
YLAIVHATNSQKPR

SEQ ID NO: 63 - protein, 13 aa, synthetic construct
AAWLAFHITNFDR

SEQ ID NO: 64 - protein, 20 aa, synthetic construct
AMLSGPGQFAENETNEVNFR

SEQ ID NO: 65 - protein, 13 aa, synthetic construct
ARFELLCSPLFNK

SEQ ID NO: 66 - protein, 12 aa, synthetic construct
WVQTSIRLLDQK

SEQ ID NO: 67 - protein, 18 aa, synthetic construct
YIKPQSQFIVDLGAYLEK

SEQ ID NO: 68 - protein, 14 aa, synthetic construct
YLAIVHATNSQKPR

SEQ ID NO: 69 - protein, 14 aa, synthetic construct
YLAIVLAANSMNNR

SEQ ID NO: 70 - protein, 14 aa, synthetic construct
YTTSTITSGIIIEK

SEQ ID NO: 71 - protein, 14 aa, synthetic construct
ADVSAFLRMNLFQK

SEQ ID NO: 72 - protein, 13 aa, synthetic construct
AMMKLTNSAEVAK

SEQ ID NO: 73 - protein, 14 aa, synthetic construct
ASKYFIVGLSCLYK

SEQ ID NO: 74 - protein, 15 aa, synthetic construct
VVFFAIQLPVYEQQK

SEQ ID NO: 75 - protein, 14 aa, synthetic construct
VYNTVISYIESNRK

SEQ ID NO: 76 - protein, 18 aa, synthetic construct
YIKPQSQFIVDLGAYLEK

SEQ ID NO: 77 - protein, 12 aa, synthetic construct
YRQMHGVAITYK

SEQ ID NO: 78 - protein, 13 aa, synthetic construct
AENGPGPGVLVLR

SEQ ID NO: 79 - protein, 13 aa, synthetic construct
AFCQSPSLIKHQR

SEQ ID NO: 80 - protein, 20 aa, synthetic construct
AMLSGPGQFAENETNEVNFR

SEQ ID NO: 81 - protein, 13 aa, synthetic construct
VSASTVPTDGSSR

SEQ ID NO: 82 - protein, 12 aa, synthetic construct
WVKQVVHMLSQR

SEQ ID NO: 83 - protein, 14 aa, synthetic construct
YALYDATYETKESK

SEQ ID NO: 84 - protein, 18 aa, synthetic construct
YIKPQSQFIVDLGAYLEK

SEQ ID NO: 85 - protein, 14 aa, synthetic construct
YQELPNSGPPHDRR

SEQ ID NO: 86 - protein, 13 aa, synthetic construct
AAWLAFHITNFDR

SEQ ID NO: 87 - protein, 20 aa, synthetic construct
AMLSGPGQFAENETNEVNFR

SEQ ID NO: 88 - protein, 13 aa, synthetic construct
ARFELLCSPLFNK

SEQ ID NO: 89 - protein, 13 aa, synthetic construct
YINWNFIRICNDR

SEQ ID NO: 90 - protein, 20 aa, synthetic construct
YSVTRSTGDVTSVSGHAMQK

SEQ ID NO: 91 - protein, 14 aa, synthetic construct
YVDSEGHLYTVPIR

(19) United States
(12) Patent Application Publication
(10) Pub. No.: US 2023/0194543 A1
(43) Pub. Date: Jun. 22, 2023

(54) VIRUS PEPTIDE AND PROTEIN VARIANT SELECTION WORKFLOW

(71) Applicant: Waters Technologies Corporation, Milford, MA (US)

(72) Inventors: Johannes Vissers, Breda (NL); Thomas McDonald, Holden, MA (US); Keith Richardson, High Peak (GB)

(73) Assignee: Waters Technologies Corporation, Milford, MA (US)

(21) Appl. No.: 18/053,093

(22) Filed: Nov. 7, 2022

Related U.S. Application Data
(60) Provisional application No. 63/276,783, filed on Nov. 8, 2021.

(51) Int. Cl. G01N 33/68; G01N 30/88

(52) U.S. Cl. CPC G01N 33/6848; G01N 30/88; G01N 2030/8831

(57) ABSTRACT

Provided herein are methods, techniques and processes for selecting one or more peptides for virus detection (e.g., influenza, SARS-CoV-2) in clinical samples using mass spectrometry. Methods for selecting a combination of peptides to identify one or more disease states in a patient can include: (i) selecting the one or more disease states; (ii) collecting information on proteins associated with the one or more disease states being present within the subject; (ii) in silico digesting individual proteins associated with the one or more disease state to obtain possible workflow peptides; (iv) collecting filtering data associated with the possible workflow peptides; (iv) analyzing the possible workflow peptides for coverage of the one or more disease states and for mass spectrometry detection and resolution; and (iv) selecting the combination of peptides from the possible workflow peptides based on the analyzing step.

### CROSS-REFERENCE TO RELATED APPLICATIONS

This application claims priority to and benefit of U.S. provisional patent application No. 63/276,783, filed Nov. 8, 2021 entitled “Virus Peptide and Protein Variant Selection Workflow,” the content of which is incorporated herein by reference in its entirety.

### REFERENCE TO AN ELECTRONIC SEQUENCE LISTING

The contents of the electronic sequence listing (W-4379-US02.xml; Size: 80,150 bytes; and Date of Creation: Nov. 4, 2022) is herein incorporated by reference in its entirety.

### FIELD OF THE TECHNOLOGY

The present disclosure relates to methods, techniques and processes for selecting one or more peptides for virus detection in clinical samples using mass spectrometry. More specifically, this technology relates to methods, techniques, and processes for selecting peptides for detection by mass spectroscopy that are indicative of positive infection of a disease state, such as influenza, in a subject or in a pooled sample of multiple subjects. This technology also relates to methods, techniques, and process for selecting peptides for detection by mass spectroscopy that are indicative of a number of virus protein variants.

### BACKGROUND

Seasonal influenza is caused by influenza A and B viruses which circulate globally. In temperate and many tropical and subtropical regions, season influenza viruses are typically transmitted during widespread outbreaks coinciding with winter or rainy periods. Other tropical or subtropical countries may experience prolonged seasonal influenza epidemics or year-round circulation. A hallmark of influenza viruses is their ability to undergo antigenic drift in response to population immunity. Because of antigenic drift, influenza vaccine antigen composition is reformulated regularly to match the vaccine strains as closely as possible to currently circulating influenza viruses.

Occasionally, influenza A viruses undergo antigenic shift. These changes may be caused by reassortment between different influenza A subtypes, such as between animal and human subtypes. Pandemic influenza can result if there is very limited or no immunity in the population, if sustained person-to-person transmission occurs, and if infection causes clinical illness (e.g., SARS-CoV-2).

In addition to influenza and corona viruses, other viruses, such as human rhinoviruses cause distress for the respiratory system. These viruses, while common and responsible for more than one-half of cold-like illnesses, can be particularly troublesome for patients with asthma, infants, elderly, and immunocompromised.

It is not uncommon for a human subject to be infected with multiple viruses at the same time. And during times of pandemic or at the height of flu season, treatment options and contact tracing efforts may require rapid identification of the source of a subject's symptoms. With rapidly changing viruses and variants, and the possibility of infection with multiple different viruses, detection protocols and tools are needed to be able to rapidly test and identify the source of infection.

### SUMMARY OF THE TECHNOLOGY

In general, the present technology is directed to the selection of peptides for detection by mass spectroscopy that are indicative of positive infection of a disease state, such as a viral disease state, e.g., influenza. The present technology, in some instances, can be used to determine which peptides to analyze from a protein sample to clinically determine using mass spectrometry the presence or absence of a disease state. For example, the present technology can be used to provide a workflow extending from sample preparation through mass spectrometry analysis of a single or pooled sample to analyze the presence of one or more peptides as an indication of the disease/infection state. In some embodiments, the workflow identifies two or more peptides (e.g., 2, 3, 4, 5, 6, 7, or more) that when detected together in a sample indicate the presence of the disease/infection state.

In general, the present technology can be utilized to identify a combination of peptides and even processing conditions for sample preparation and analysis of the selected peptides for an efficient mass spectrometry based workflow for the detection of a diseased state providing broad coverage across different variants of disease. The present technology can be conducted in silico without the need for performing trial and error experimentation in a mass spectrometer. In some embodiments, the present technology is directed to the selection of peptides and creation of a workflow based on the selected peptides for a yes/no result in a clinical analysis of a disease state of an individual subject. In certain embodiments, the present technology encompasses the selection of peptides and creation of a workflow for a yes/no result in a clinical analysis of a disease state present in a pooled sample (i.e., a sample including digested proteins from a number of subjects pooled together). In other embodiments, the present technology can be utilized to determine the workflow for detecting if one or more of multiple, different disease states (e.g., the presence of more than one virus or infection, such as, SARS-CoV-2 and/or influenza) in a subject. The technology could be used to select workflow peptides and create workflows that indicate the presence or absence of the one or more virus or infection (e.g., a simple yes that at least one virus or infection is present, or no virus or infection is detected). In other embodiments the technology could be utilized to select workflow peptides and create workflows that indicate, if a virus or infection is detected, which virus variant (or variant group) or infection is detected.

Some embodiments of the present technology include collection steps followed by one or more selection steps to select peptides for the particular disease/viral state and then to create the workflow for efficient mass spectrometry detection of the disease state. The collection steps can include use of knowledge databases to identify known or partially known protein sequences of specific viruses, such as influenza. From this collected information, appended with meta data, the primary protein amino acid sequence can be used to conduct in silico tryptic digestion of the proteins to form possible workflow peptides. Ionization efficiencies, retention times, and/or other physicochemical properties for the possible workflow peptides are then determined (e.g., calculated using algorithms based upon physicochemical properties of the peptides and proteins) and added to the collection of information. Using the data (both collected and calculated) from these initial collection steps, selection steps (e.g., filtering) are then applied to select the actual workflow peptides.

Selection steps can include selecting the peptides and proteins based on one or more physicochemical properties and/or homology. For example, length of peptides, amino acids or amino acid motif of the possible workflow peptides, retention time of the possible workflow peptides in LC, and ionization and separation of the possible workflow peptides in a mass spectrum can be used to filter (potential elimination of some, increase importance of others) possible workflow peptides. Further selection steps can also be applied to filter data for the selection of workflow peptides. For example, a frequency (% of sequencing containing a given peptide) filter can be applied to analyze the number of peptides that are shared between strains and types of disease states (e.g., strains and variant types of influenza or other virus) and/or filtering based on available meta data associated with the protein (variants) of interest. This filter can also be used to assess a minimal number of peptides to find the largest number of indicative (virus) proteins of the disease state to monitor.

In a final selection step, statistical analysis, such as Markov Chain Monte Carlo techniques, genetic algorithms, Bayesian Inference, nested sampling or other statistical analysis tools including those employed in Bayesian Inference, can be applied to the filtered possible workflow peptides, to select the actual workflow peptides for determining the broadest variant coverage of the selected disease state (e.g., for the yes/no determination of influenza). Once the actual workflow peptides have been identified (e.g., coverage determined), the physicochemical properties of these peptides together with information from the in silico tryptic digestion for those actual workflow peptides can be provided for use in the final workflow (i.e., sample preparation (digestion, clean-up etc.) sample separation (liquid chromatography) and mass spectrometry (ionization conditions and m/z information). In other embodiments, once the actual workflow peptides have been identified/selected, the values for a given set of experimental conditions will be calculated based upon particular equipment and experimental set-up to be utilized as well as other techniques known to those of ordinary skill in the art.

In some embodiments, once the workflow has been formed using the methods and processes of the present technology, external resources and expert input (e.g., virologists) can be used for validation.

One aspect of the technology is directed to a method for selecting a combination of peptides to identify one or more disease states in a subject using mass spectrometry. The method includes: selecting the one or more disease states; collecting information on proteins associated with the one or more disease states being present within the subject; in silico digesting individual proteins associated with the one or more disease state to obtain possible workflow peptides; collecting filtering data (e.g., physicochemical data, homology data, abundance of peptide data, data associated with the one or more disease states, any combination of the foregoing, etc.) associated with the possible workflow peptides; analyzing the possible workflow peptides for coverage of the one or more disease states and for mass spectrometry detection and resolution; and selecting the combination of peptides from the possible workflow peptides based on the analyzing step.

The above aspect can include one or more of the following features. In some embodiments, the method features selecting two or more disease states (e.g., two disease states, three disease states, four disease states, etc.). In certain embodiments, the method features selecting two or more variants of the one or more disease states. In some embodiments, the filtering data comprises physicochemical data. The physicochemical data can comprises ionization efficiency data and/or retention time data. In some embodiments, the physicochemical data comprises one or more of (a) length of possible workflow peptides; (b) MRM transition data on co-eluting or close eluting possible workflow peptides; and (c) amino acid sequences contained within the possible workflow peptides. In some embodiments, the filtering data comprises data on methionine being present within the amino acid sequences contained within the possible workflow peptides. Some embodiments of the method can further include eliminating possible workflow peptides using the filtering data prior to analyzing the possible workflow peptides for coverage. Other embodiments can further include eliminating possible workflow peptides after analyzing the possible workflow peptides for coverage. The step of analyzing the possible workflow peptides for coverage can include applying a statistical approach, such as, for example Bayesian inference or Markov Chain Monte Carlo algorithm. In some embodiment, the step of analyzing the possible workflow peptides for coverage includes analyzing coverage for a yes/no result for the disease state. In certain embodiments, the step of analyzing the possible workflow peptides for coverage includes analyzing coverage for a determination of a particular variant of the disease state (e.g., Influenza A versus Influenza B). In some embodiments, the step of analyzing the possible workflow peptides for coverage includes analyzing coverage for a determination of a particular disease state from a group of possible disease states (influenza versus SARS-CoV-2 versus RSV).

In general, the present technology for the selection of peptides for determination of the presence or absence of a disease state through mass spectrometry provides many advantages. For example, the present technology can be used to capture, calculate, and evaluate hundreds of thousands of possible peptides for identification of a particular disease state. By applying the collection and selection techniques to the calculated data, efficient mass spectrometry based workflows can be generated to positively identify a disease state. The technology eliminates the trial and error timeline and the costs of conducting laboratory testing to identify and select appropriate surrogate peptides that are indicative of a disease state and can also be easily resolved and/or processed in an efficient workflow.

### BRIEF DESCRIPTION OF THE DRAWINGS

The technology will be more fully understood from the following detailed description taken in conjunction with the accompanying drawings, in which:

FIG. 1A is a schematic of an embodiment of a workflow in accordance with the present technology.

FIG. 1B is a schematic of another embodiment of a workflow in accordance with the present technology.

FIG. 2 is a schematic illustrating the types, subtypes, lineages, clades and sub-clades of human seasonal influenza viruses.

FIG. 3 is a schematic illustrating filtering and coverage steps applied for the selection of peptides in accordance with an embodiment of the present technology.

FIG. 4A provides an example peptide selection results (for solution 1, 65 peptides) in virtual chromatographic form obtained using different random seeds resulting from MCMC processing of the pre-filtered collected information.

FIG. 4B provides an example peptide selection results (for solution 2, 65 peptides) in virtual chromatographic form obtained using different random seeds resulting from MCMC processing of the pre-filtered collected information.

FIG. 4C provides an example peptide selection results (for solution 3, 66 peptides) in virtual chromatographic form obtained using different random seeds resulting from MCMC processing of the pre-filtered collected information.

FIGS. 5A-5F provide three example peptide selection results in virtual chromatographic form for all UniProt Protein Knowledge Database influenza viruses. The top three panels (FIG. 5A, FIG. 5C, and FIG. 5E) represent the 100% variant detection coverage embodiment; whereas the bottom three panels (FIG. 5B, FIG. 5D, and FIG. 5F) are the 95% embodiment. FIG. 5A and FIG. 5B illustrate cases where all protein variants are considered; FIG. 5C and FIG. 5D illustrate embodiments where only the high frequently occurring virus genes/proteins are considered; and FIG. 5E and FIG. 5F illustrate the embodiment in which genes/proteins present are the ones that can undergo mutation, gene translation are considered.

FIG. 6A is a schematic showing both (i) the experimental conditions of an in silico determination using influenza A and B data and (ii) a representative portion of sequences identified in each of three possible solutions for 100% variant coverage. FIG. 6A recites SEQ ID NO 1; SEQ ID NO 2; SEQ ID NO 3; SEQ ID NO 4; SEQ ID NO 5; SEQ ID NO 6; SEQ ID NO 7; SEQ ID NO 8; SEQ ID NO 9; SEQ ID NO 10; SEQ ID NO 11; SEQ ID NO 12; SEQ ID NO 13; SEQ ID NO 14; SEQ ID NO 15; SEQ ID NO 16; SEQ ID NO 17; SEQ ID NO 18; SEQ ID NO 19; SEQ ID NO 20; and SEQ ID NO 21.

FIG. 6B is a schematic showing both (i) the experimental conditions of an in silico determination using influenza A and B data and (ii) a representative portion of sequences identified in each of three possible solutions for 95% variant coverage. FIG. 6B recites SEQ ID NO 22; SEQ ID NO 23; SEQ ID NO 24; SEQ ID NO 25; SEQ ID NO 26; SEQ ID NO 27; SEQ ID NO 28; SEQ ID NO 29; SEQ ID NO 30; SEQ ID NO 31; SEQ ID NO 32; SEQ ID NO 33; SEQ ID NO 34; SEQ ID NO 35; SEQ ID NO 36; SEQ ID NO 37; SEQ ID NO 38; SEQ ID NO 39; SEQ ID NO 40; SEQ ID NO 41; and SEQ ID NO 42.

FIG. 7A is a graphical representation of a combination of peptides selected to achieve a certain protein or protein complement virus variant detection coverage. FIG. 7A includes SEQ ID NO 43; SEQ ID NO 44; SEQ ID NO 45 and SEQ ID NO 46.

FIG. 7B is an example of the magnitude of the coverage that can be achieved by selecting an appropriate combined number of peptides for all UniProt Protein Knowledge Database influenza virus proteins.

FIG. 8A is a schematic showing both (i) the experimental conditions of an in silico determination using RSV data and (ii) a representative portion of sequences identified in each of three possible solutions for 100% variant coverage. FIG. 8A recites SEQ ID NO 47; SEQ ID NO 48; SEQ ID NO 49; SEQ ID NO 50; SEQ ID NO 51; SEQ ID NO 52; SEQ ID NO 53; SEQ ID NO 54; SEQ ID NO 55; SEQ ID NO 56; SEQ ID NO 57; SEQ ID NO 58; SEQ ID NO 59; SEQ ID NO 60; SEQ ID NO 61; SEQ ID NO 62; SEQ ID NO 63; SEQ ID NO 64; SEQ ID NO 65; SEQ ID NO 66; SEQ ID NO 67; SEQ ID NO 68; SEQ ID NO 69; and SEQ ID NO 70.

FIG. 8B is a schematic showing both (i) the experimental conditions of an in silico determination using RSV data and (ii) a representative portion of sequences identified in each of three possible solutions for 95% variant coverage. FIG. 8B recites SEQ ID NO 71; SEQ ID NO 72; SEQ ID NO 73; SEQ ID NO 74; SEQ ID NO 75; SEQ ID NO 76; SEQ ID NO 77; SEQ ID NO 78; SEQ ID NO 79; SEQ ID NO 80; SEQ ID NO 81; SEQ ID NO 82; SEQ ID NO 83; SEQ ID NO 84; SEQ ID NO 85; SEQ ID NO 86; SEQ ID NO 87; SEQ ID NO 88; SEQ ID NO 89; SEQ ID NO 90; and SEQ ID NO 91.

### DESCRIPTION OF THE TECHNOLOGY

In general, the present technology is directed to the selection of peptides for detection by mass spectroscopy that are indicative of a disease state (e.g., influenza infection, corona virus infection, human rhinovirus infection, etc.). The present technology provides processes in which data are first collected and/or calculated and then filtered using physicochemical properties and other characteristics and traits of the diseased state to provide an efficient workflow for the mass spectrometry detection of the diseased state in a sample.

One challenge associated with efficiently diagnosing an influenza infection is the variability that exists among influenza types and subtypes. Due to this variability, reagents and methods designed to detect one type or subtype of the virus may not detect an infection with a different influenza type of subtype. Three types of influenza viruses infect human subjects: influenza A, influenza B, and influenza C. Influenza A and B are typically associated with seasonal flu, while influenza C generally causes mild disease. A fourth type, influenza D, can infect certain non-human mammals such as cattle. Influenza viruses are further divided into subtypes/strains based on the composition of surface proteins that make up the viral capsid. For influenza A, the subtypes are based on the expression of particular variants of the surface proteins hemagglutinin (H) and neuraminidase (N). There are 18 known different variants of hemagglutinin (H1-H18), and 11 known different variants of neuraminidase (N1-N11), combinations of which result in 198 possible different influenza A subtypes (over 130 of which have been detected in nature). In view of the prevalence of influenza infection, and the overlap of influenza symptoms with other common pathogenic infections (e.g., SARS-CoV-2 infection, human rhinovirus infection), methods of accurately and efficiently detecting and diagnosing viral infection are needed.

One objective of the present technology is to identify a set of peptides that can be used to detect the presence of an influenza virus in a sample (e.g., a biological sample such as nasal swab, saliva, sputum, blood, plasma, etc.), derived from a subject known or suspected of having an influenza infection. In exemplary embodiments, the set of peptides can include peptides having sufficient homology commonality among influenza types or subtypes to allow detection of multiple influenza types or subtypes. In addition, in some embodiments, the peptides have minimal to no homology with non-influenza proteins, thereby serving as specific marker(s) of influenza infection. By way of example, the present technology can be used to identify one or more (e.g., two or more, three or more, four or more, five or more, six or more, seven or more, eight or more, nine or more, ten or more, etc.) peptides that comprise a sequence that is shared among one or more influenza types and/or one or more influenza subtypes. In some embodiments, the present technology can be used to identify peptides that have significant homology (i.e., having at least 80%, at least 85%, at least 90%, at least 95%, at least 97%, at least 99%, or 100% sequence identity) in two or more influenza types (e.g., peptides that have significant homology in influenza A and influenza B). In some embodiments, the present technology can be used to identify peptides that have significant homology (i.e., having at least 80%, at least 85%, at least 90%, at least 95%, at least 97%, at least 99%, or 100% sequence identity) in two or more influenza subtypes (e.g., peptides that have, for example, significant homology in influenza A H1N1 and influenza A H3N2). In some embodiments, the technology provides a set of peptides, wherein each peptide in the set of peptides has significant homology in two, three, or four influenza types selected from influenza A, influenza B, influenza C, and influenza D, or even novel, mutated variants. In some embodiments, the technology provides a set of peptides, wherein each peptide in the set of peptides has significant homology in two or more, three or more, four or more, five or more, six or more, seven or more, eight or more, nine or more, or ten or more influenza subtypes. In some embodiments, each peptide in the set of peptides has minimal homology (e.g., 60% or less, 50% or less, 40% or less, 30% or less, 20% or less, or 10% or less sequence identity) to non-influenza peptides or proteins. For example, in some embodiments, each peptide in the set of peptides can have minimal homology to a human protein, and/or a protein of animal origin, e.g., a canine protein or a feline protein. Selection of peptides in accordance with the methodology described herein can be used to identify a minimal set of peptides that can be used to detect multiple types or subtypes of influenza in a sample, e.g., a patient sample.

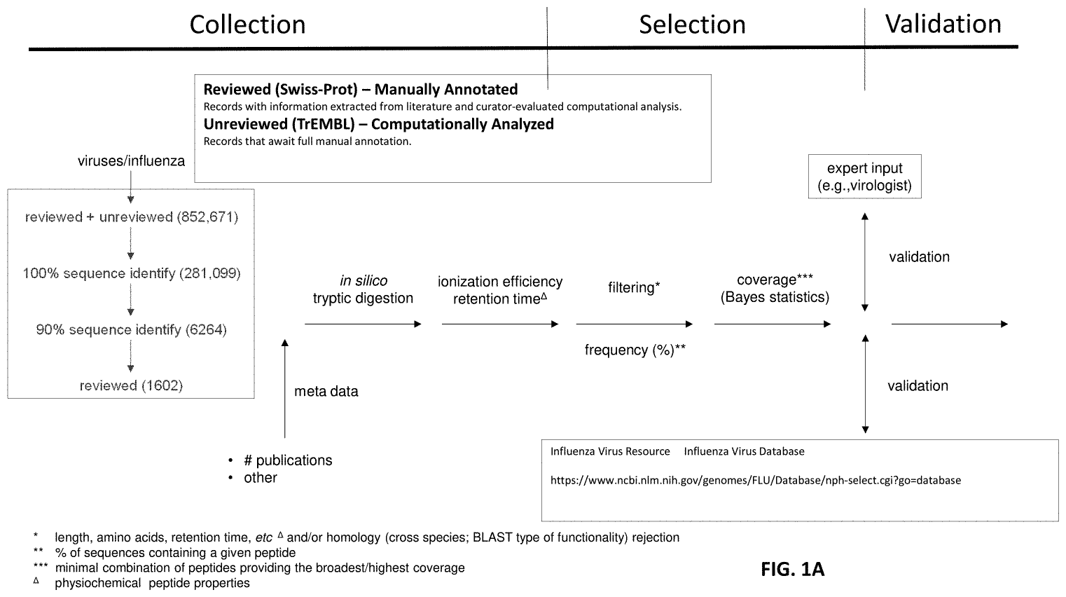

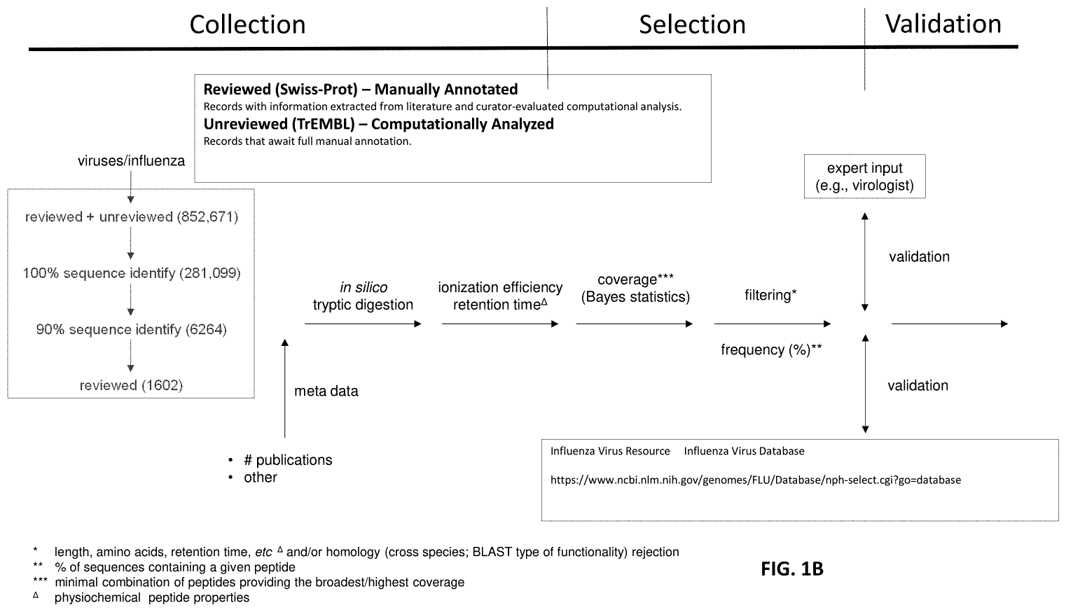

In general, processes of the present technology include collection/calculation steps and selection/weighting steps. FIG. 1A illustrates an embodiment of the overall process and provides an overview of the division of collection versus selection steps. FIG. 1B illustrates an alternative embodiment, in which the order of the selection steps differs from the process shown in FIG. 1A. Also shown in both FIGS. 1A and 1B, but is an optional step, is a validation step, which can use one or more of expert input or external resources (e.g., virologists) to validate the workflow created from using the processes of the present technology.

As an initial matter, processes of the present technology begin with the identification of the viruses or disease states for detection in a clinical analysis as well as the type of information required for a result from the clinical analysis. For example, in one embodiment, the virus to be detected is influenza in humans, and the result desired from the clinical analysis is a simple yes/no for any type of seasonal influenza infection.

As described above, there are 4 types of influenza virus (types A, B, C, and D). Only types A-C are known to infect humans. Type D primarily affects cattle. Further, type C infections generally result in mild symptoms. Thus, type C is generally not considered to be associated with seasonal pandemic outbreaks. As the present embodiment is directed to detecting influenza in human (and more particularly to harmful influenza), the result of the clinical analysis of the sample would indicate the presence or absence of an influenza infection (either or both type A/type B) in a sample from the human subject. In other embodiments, the workflow need not be restricted to variants A and B. In still yet further embodiments, the workflow is directed to other viruses or other viruses in combination with influenza (e.g., human rhinovirus and/or SARS-CoV-2).

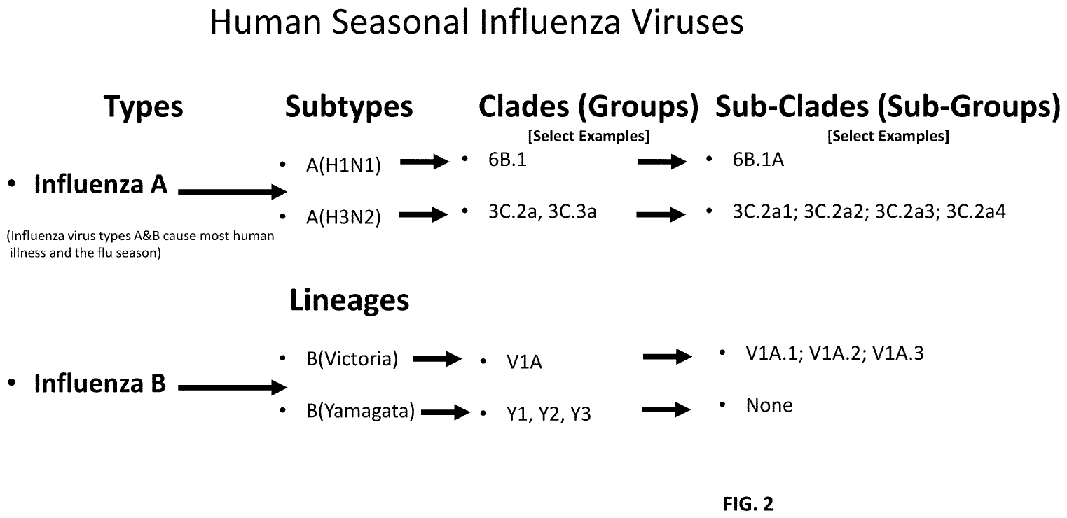

Influenza viruses can be further categorized by subtype (for type A) or lineage (for type B). Additional information and classification of the virus can be discerned through genetic clades and sub-clades as shown in FIG. 2. In general, viruses comprise proteins as part of their biomolecular complement. These proteins are typically indicative of or provide information useful for the detection of the disease state in a sample. Further, while it is the surface proteins that are typically used in characterization of these viruses, as they are typically involved in detection by the host, and therefor form potentially suitable drug candidates, detection or identification of the entire protein complement of viral particles is challenging and time consuming. The process of breaking down the proteins (generally by enzymatic digestion) creates so-called surrogate peptides that can be better resolved and detected by mass spectrometry.

Returning to FIG. 1A, the process shown provides a method for the selection of surrogate peptides to detect by mass spectrometry for the diagnosis of a disease state. In particular, the method shown in FIG. 1A is directed to the selection of workflow peptides and corresponding workflow for the diagnosis of a viral infection (e.g., influenza). After having identified influenza in this particular example as the disease state, the method begins with the first collection step shown on the left side, in the shaded box. Specifically, this first collection step involves using data bases, such as for example, the UniProt protein knowledge database (www.uniprot.org), the NCBI Virus Community Portal (ncbi.nlm.nih.gov/labs/virus/vssi/#/), and the GISAID Initiative (www.gisaid.org). For example, in this first collection step, databases are accesses to collect information on the proteins associated with each type, subtype, lineage, clades, and sub-clades of the virus. As shown in this first step (the shaded box), information about these proteins come from reviewed and unreviewed sources, and from information in which the sequence of the proteins are 100% or at least 90% homologues with other protein database entries and treated as so-called clusters or groups. Reviewed information is typically manually annotated and includes records with information extracted from literature and curator-evaluated computational analysis. Unreviewed information is typically computationally analyzed, and includes records that await full manual annotation.

From this first step of collecting data relating to the proteins associated with viral infection (e.g., influenza), additional meta data is collected on each of the proteins. The meta data is then associated with the in silico tryptic digestion of the proteins identified in the first collection step to determine the possible workflow peptides. (See Beynon, R. J.; Bond, J. S.; Proteolytic Enzymes: A Practical Approach, 2^{nd} Edn. Oxford University Press, Oxford, U K 2001, pp. 149-183). Having access to this list, further calculations using the physicochemical properties of the peptides are computed to determine their ionization efficiencies and other peptide characteristics (for mass spectrometry) and retention times (for LC separation prior to MS). For example, Geromanos et al., in “Simulating and validating proteomics data and search results” Proteomics 2011, 11, 1189-1211 describe an in-silico method for the generation proteomes, which utilizes the underlying physicochemical properties of peptides and proteins to compute peptide characteristics. Additionally, M. Gilar et al. describe various prediction models for peptide separation in their published paper entitled “Utility of retention prediction model for investigation of peptide separation selectivity in reverse-phase liquid chromatography: impact of concentration of trifluoroacetic acid, column temperature, gradient slop and type of stationary phase” published in Analytical Chemistry, Vol. 82, No. 1, Jan. 1, 2010 and also in M. Gilar et al. “Solvent selectivity and strength in reversed-phase liquid chromatography separation of peptides” published in Journal of Chromatography A, 1337, (2014), pages 140-146. All of this information is calculated and collected so that it can be used in the selection/weighting steps applied in the second stage of the process shown in FIGS. 1A and 1B.

As mentioned above, the processes of the present technology include both collection/calculation steps followed by selection/weighting steps. In the selection stage of the process, the information calculated and collected on possible workflow peptides is evaluated and ranked to arrive at actual workflow peptides for viral detection using mass spectrometry.

After calculation and collection of ionization efficiency and retention times, as well as other physicochemical properties, filtering/weighting based on physicochemical properties and homology determination begins. Specifically, using information regarding the length of the peptides, the amino acid sequences therein (i.e., their motif), retention times to determine co-eluting or near eluting (isobaric) peptides, and MRM transitions of the co-eluting or near eluting peptides, are evaluated and used to discount (i.e., filter out, down score, reduce weighting, etc.) at least a portion of the possible workflow peptides generated from the previous collection and calculation steps. For example, the length of the possible workflow peptides is evaluated. Possible workflow peptides having a longer length are disfavored due to difficulties in liquid chromatography or mass spectroscopy analysis. Therefore, a threshold cut-off is applied as a filter (e.g., peptides having a sequence length longer than 15 amino acids are eliminated in this example). Other criteria include retention time (e.g., selecting peptides that elute at different times) and/or MRM transition differences, i.e., non-isobaric peptides and/or MRM transitions, should the peptides co-elute and potentially give rise to detection interference.

In addition to the physicochemical properties and homology filter criteria described above, other filtering or scoring functions can be applied. For example, the frequency % of a peptide (i.e., the % protein amino acid of sequences containing a given peptide) can be used to generate a score function for selecting possible workflow peptides. As a simple example, a score function (scaled between 0 and 1) is calculated by multiplying the particular possible workflow peptide frequency by the number of publications describing the protein function the peptide is associated with. As a result, peptides that are common between strains and types of influenza (high frequency) that are described extensively can be taken into account, such that a minimal number of possible workflow peptides can be selected as a way to positively detect one or more proteins indicative of viral infection.

Using one or more of physicochemical properties, homology, and/or frequency scoring factors, a scoring-function, resulting from the inference analysis and accumulating to a comprehensive, a final, multi-factorial, weighted score can be applied to filter the results. In some embodiments, instead of using a scoring factor to capture all aspects, a more simplistic threshold cut-off can be applied, such as eliminating all possible workflow peptides having more than 15 amino acids. In other embodiments, the scoring factor can be refined to capture all aspects used in the selection steps (e.g., used in addition to or in replacement of cut-off values). Other ways of calculating a scoring function or different scoring functions are also within the scope of the preset technology. For example, some data can be excluded from the analysis due to expert or domain knowledge regarding sample preparation, behavior/properties, and detection of certain peptides. For example, peptides that contain the amino acid methionine (M), an amino acid with a hydrophobic side chain, are particularly challenging to resolve as they may reside in two forms, in an oxidized and non-oxidized form. Thus, using filtering/weighting steps of the present technology, these peptides can be excluded.

In one embodiment, a Bayesian statistics based analysis is utilized for the final selection of the actual workflow peptides. Once the actual workflow peptides are determined, the data used in modeling these actual peptides through the in silico tryptic digestion, ionization efficiency and retention time, and other meta data is provided, such that the workflow for the mass spectrometry detection of all actual workflow peptides can be reviewed and applied.

In another embodiment, a Markov Chain Monte Carlo (MCMC) algorithm is utilized for targeting peptides for selection of the actual workflow peptides. MCMC is a method that allows for the efficient exploration of high-dimensional probability distributions by obtaining random samples (Monte Carlo) from the distribution using an iterative process in which each iteration depends only on the properties of the distribution at the current position and possible destinations (Markov Chain). This method can be used to limit the enormous number of possible experiments in checking each individual target list of peptides to arrive at the selected or actual workflow peptides.

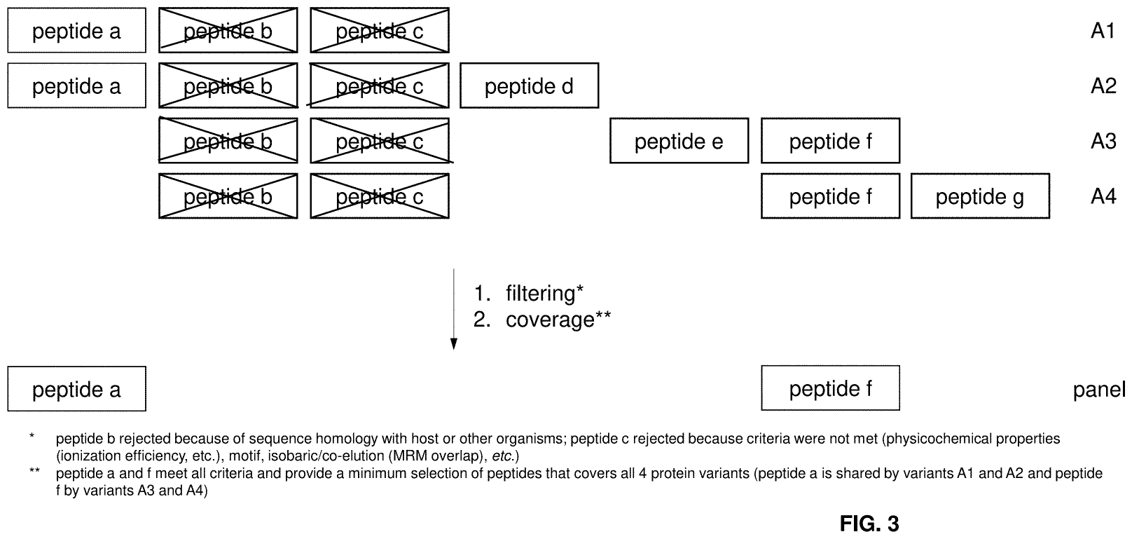

FIG. 3 provides an illustration of the selection steps shown in FIGS. 1A and 1B. In the embodiment shown in FIG. 3, there are 4 protein variants (A1, A2, A3, and A4). After the calculations of the possible workflow peptides from the in silico digestion modeling, peptide a, peptide b, peptide c, peptide d, peptide e, peptide f, and peptide g were identified as possible workflow peptides. As can be seen in FIG. 3, variant A1 after digestion produces peptide a, peptide b, and peptide c. Variant A2 after digestion produces peptide a, peptide b, peptide c, and peptide d. After digestion of variant A3, peptide b, peptide c, peptide e and peptide e are generated. And digestion of variant A4 produces peptide b, peptide c, peptide f, and peptide g. Thus, in this illustration, the possible workflow peptides are peptide a, peptide b, peptide c, peptide d, peptide e, peptide f, and peptide g. Both filtering and coverage (both part of the selection steps shown in FIGS. 1A and 1B) are applied to filter (eliminate/apply a low weighting score) possible workflow peptides in an effort to get to the selection process of actual workflow peptides for mass spectrometry analysis for a clinical diagnosis of the presence or absence of at least one of variant A1, variant A2, variant A3, and variant A4 in a sample. Applying the first type of selecting/weighting, physicochemical/homology criteria, peptide b is eliminated because of sequence homology with a host of other organisms (making its detection a-specific). In addition, peptide c is eliminated because of certain physicochemical properties are not advantageous to providing an efficient workflow. For example, the ionization efficiency may be too low and or co-elution with other relevant peptides may result in MRM interference, making detection challenging for an advantageous workflow.

After eliminating peptide b and peptide c using the filtering methods described above, statistical analysis regarding the coverage of detection of the four variants with the least amount of peptides is undertaken to make the final selection of the actual workflow peptides. In the illustration provided in FIG. 3, we see that peptide a covers both variants A1 and A2, and peptide f covers both variants A3 and A4. As a result, the most efficient workflow for the detection of at least one of variants A1, A2, A3, and A4 in a sample is to select peptide a and peptide f as the actual workflow peptides and eliminate all others.

As mentioned above, FIG. 1B illustrates an alternative workflow. In the process shown in FIG. 1B (selection stage), the statistical inference analysis is conducted first, followed by filtering/weighting using user-defined criteria. The user-defined filtering/weighting is optional. One of the outcomes of the statistical inference analysis could be a scoring function (down-weighting the selection of certain peptides). A benefit of this approach is that peptides are not excluded (filtered out) during any part of the analysis.

The process of the present technology can optionally include a final step after the collection/computational steps and selection steps. In the embodiments shown in FIGS. 1A and 1B, after the selection of the actual workflow peptides, a validation step utilizing expert input (e.g., virologists) and/or external resources, such as NIH databases (e.g., www.ncbi.ntm.hih.gov/genomes/FLU/Database/nph-select.cgi?go=database) can be used to further validate the combination of selected actual workflow peptides and/or the sample preparation and MS workflows used in modeling these actual workflow peptides.

The above illustrations of the present technology are directed to obtaining a Yes/No result of virus detection from a single subject. However, the present technology is not limited to just a single class of infection, such as, for example, influenza. For example, the present technology can be applied to a clinical analysis (a mass spectrometry based analysis) of a sample to determine if more than one virus is present in a single sample. That is, the processes of the present technology can be utilized to select actual workflow peptides for a MS determination of whether the subject is infected with SARS-CoV-2, influenza (type A or type B, or novel variant) and/or any other coronavirus or season viral infection (e.g., human rhinovirus). In addition, the present technology is also applicable to providing a more detailed result (i.e., not just Yes or No). For example, the present technology can be used to select the actual workflow peptides which MS presence will indicate not just the presence of an influenza infection, but also whether the subject is infected with type A or type B, and potentially which variant (e.g., which subclass, etc. by incorporating information regarding the variant of interest in the coverage selection steps).

Further, the present technology is not limited to detection of viral infections. For example, the processes of the present technology can be used to select actual workflow peptides for the detection of metabolic diseases (e.g., Gaucher disease, phenylketonuria) or other types of disorders that can be detected using clinical LC/MS.

While the above illustrative embodiments were directed to a sample of a single subject, the present technology can also be employed to detect infection in pooled samples (samples stemming from the collection/pooling of numerous subjects to form a pool of subjects). Pooling can be useful when testing large sections of a population for the presence of a disease state. By pooling a number of individuals together, the hope is that a greater segment of the population can be tested regularly and discounted from having the infection, thus saving resources.

In embodiments of the technology, statistical Bayesian Inference analysis is utilized to identify target peptides that represent the protein variants of interest (e.g. coverage). Technology of the present invention (see FIGS. 1A and 1B) include collection and selection steps. Following an initial information collection phase (e.g., primary amino acid sequence protein variant and meta data), the protein virus variants are digested in silico to form proteolytic peptides for which physicochemical properties are determined such as retention time, peptide intensity (measure for ionization efficiency), product ion spectra with associated intensities (measure for fragmentation efficiency). In some embodiments, this process is followed by an optional filtering step based on user specified criteria, such as peptide length, cross species homology, and others (e.g., detection data), before modelling of the data is conducted to answer the question of identifying target peptides for a positive variant detection. That is, how to arrive at a set of N candidate target peptides that represent the protein variants of interest.

For example, each peptide from a candidate list of peptides can be either excluded or included in an experiment for arriving at or determining the selection. There are therefore 2^{N} possible experimental target lists Lᵢ where 1≤i≤2^{N} that provide varying degrees of protein variant coverage C(i) where 0<Cᵢ<1 and when C(i)=1, 100% coverage is achieved. In order to assign relative merit to these potential target lists, other constraints must be considered, such as peak capacity of the separation system and duty cycle of the MS/MS detection system along with the requirement to minimize the number of peptides utilized. In reality, because there is an enormous number of possible experiments, the target lists cannot all be checked individually (to arrive at selection of actual workflow peptides). And instead, embodiments of the present technology apply a statistical approach in the selection step of the method.

In some embodiments, such as some of the embodiments discussed above, one or more statistical approaches may be used, such as those employed in Bayesian inference. Other approaches are also possible. For example, a Markov Chain Monte Carlo (MCMC) algorithm can be utilized in the selection step of the present technology. MCMC methods allow for efficient exploration of high-dimensional probability distributions by obtaining random samples (Monte Carlo) from the distribution using an iterative process in which each iteration depends on the properties of the distribution at the current position and possible destinations (Markov Chain).

When employing a MCMC approach in the present technology, the figure of merit, which must simultaneously encompass the goals of increasing protein coverage, minimizing the number of peptides and experimental compatibility, is not strictly a probability as such and there is considerable freedom in how to define it. As a result, in some embodiments, the figure of merit is split into two concepts: a “likelihood” function which is the protein coverage C(i) raised to some power S and an exponential “prior probability distribution” for the number of peptides in the target list having a mean that controls the relative importance of this parameter. The experimental constraints are incorporated by imposing a maximum on the number of simultaneously eluting target peptides in the method. For a given target list Lᵢ this can be calculated by producing a scoring function in the form of a “virtual chromatogram” for the target list using the retention time of the targeted peptides and the system peak capacity.

In some embodiments the MCMC sampling of the included/excluded statuses of peptides may comprise Gibbs sampling, or Metropolis-Hastings sampling.

In some embodiments, the objective is to uniquely identify as many proteins as possible using a minimal number of peptides. However, owing to similarity and redundancy in the list of proteins provided, it may not be possible to find peptides that can uniquely identify certain proteins. It is therefore useful to introduce a numerical measure of “degeneracy” that can be included in the figure of merit employed in the optimization process. To give some examples, “degeneracy” could be the average number of proteins identified by a peptide sequence, the variance of this quantity or some combination of these.

In line with standard Bayesian analysis, certain embodiments employ a “posterior probability distribution” which is given by the product of the “prior probability distribution” with the “likelihood” function. In the early stages of the statistical analysis, the “likelihood” is softened (a procedure often referred to as simulated annealing) to reduce the probability of the analysis becoming trapped in local minima.

Another advantage of utilizing MCMC based approaches in selection steps is their ability to provide many alternative solutions to the problem, either by re-running the analysis several times with different random seeds, or by taking several representative samples in the final stages of the analysis. This provides some flexibility in the event that a promising looking method performs less well in practice than is predicted through simulation. To give just one example, actual retention times of peptides will differ from simulated values, which could lead to the experimental capacity being exceeded.

In some embodiments, several explorations may be carried out in parallel, and the individual exploration “objects” may interact with each other at certain times during the exploration, for example, in a genetic algorithm, nested sampling or a particle swarm optimization.

The following Examples illustrate a MCMC approach utilized in the methods of the present technology during the selection steps. Prior to the selection steps, collection steps including in silico digestion were performed.

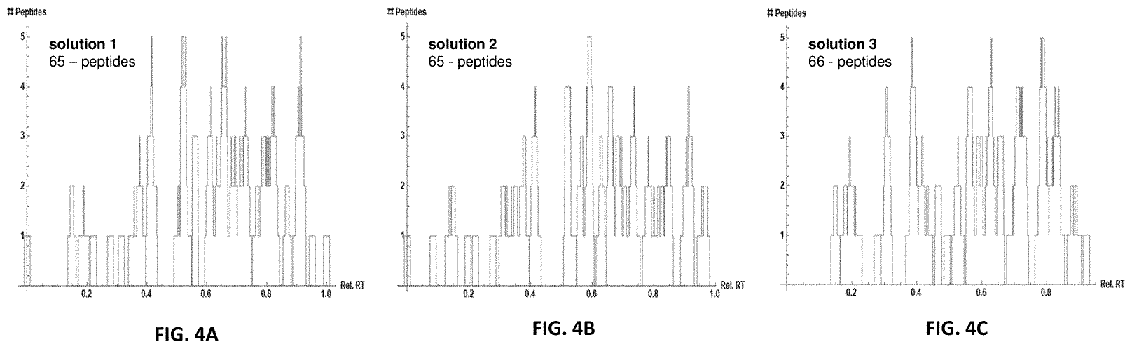

Example 1: Selection of Peptides for Differentiation of Protein Virus Variants—Analysis Using MCMC Approach. In this example, initial selection criteria were simulated to identify actual workflow peptides for the differentiation analysis of known human coronaviruses (e.g., SARS-CoV-2, SARS-CoV MERS-CoV, Coy 229E, CoV OC43, CoV NL 63, and CoVHKU1). The protein virus variant amino acid sequences were obtained from the UniRef section (100% sequence identity clusters) of the UniProt Protein Knowledge Database and processed using the collection steps illustrated in FIG. 1B. The filtering step included limiting the in silico generated peptides (digestion) to a subset of peptides with a sequence length of 5 to 15 amino acids and excluding methionine (M) containing peptides. In silico determined physicochemical analyte properties included normalized retention time and relative ionization efficiency (predicted abundance of double (2+) and triply (3+) charged peptide ions of over 10% of total abundance). FIG. 4A, FIG. 4B and FIG. 4C show three example peptide selection results obtained using different random seeds resulting from MCMC processing of the pre-filtered collected information. FIG. 4A shows the results for solution 1. FIG. 4B shows the results for solution 2. And FIG. 4C shows the results for solution 3. The results are given in the form of a virtual chromatogram (score function). In this example, the LC peak capacity (PC) was set to 50, a maximum of ten co-eluting peptides was permitted, assuming two MRM transitions per peptide to create a targeted LC-MS/MS method for differentiation (i.e., identification of individual variants, by minimizing degeneracy and consequently ensuring that the selected peptides are as unique as possible) and requiring 100% species specific variant coverage. 77% of the target peptides map to a single protein. In total, in this example 77 proteins map to 24 protein clusters (gene encoded proteins) across the different human corona viruses. 18 protein clusters can be fully resolved based on sequence unique target peptides, two cannot be resolved, and 7 partially resolved. At the protein level, 56 out 77 proteins can be fully resolved/detected based on target peptide sequence unique information.

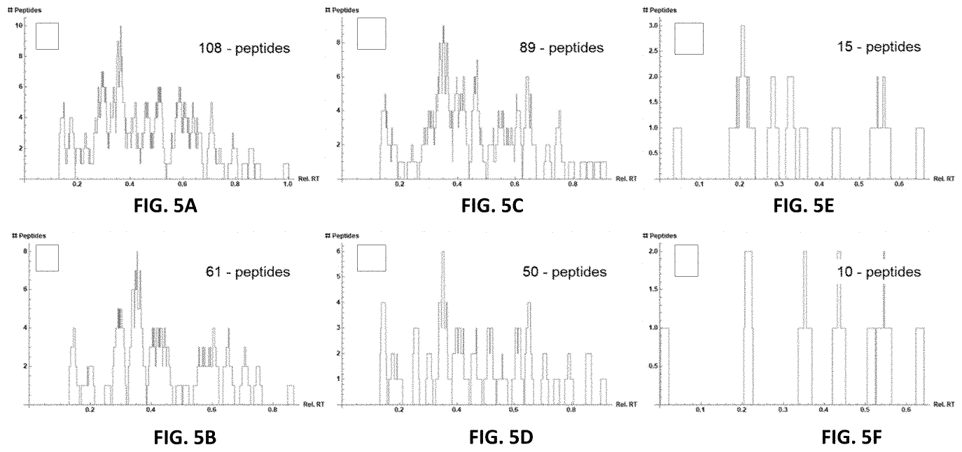

Example 2: Selection of Peptides for Detection of all Known Protein Virus Variants. In this example, the targeted detection is the presence or absence of a disease state, specifically influenza. That is, is a protein complement of Influenza A or B detected. This method can be adopted for other disease states, such as the presence of a corona virus protein variant(s). In the present example, reviewed (i.e., manually annotated) amino acid sequences were obtained from the UniProt Protein Knowledge Database, were analyzed and pre-processed using the same filter criteria as described in Example 1. FIGS. 5A-5F show the peptide selection results of the MCMC processing of the pre-filtered collected information in the form of a virtual chromatogram (score function). In this example, the PC value was set to 50, a maximum of ten co-eluting peptides were allowed, and two MRM transitions were assumed per peptide to create a targeted LC-MS/MS method for the detection of all known variants. In silico determined physicochemical analyte properties included normalized retention time and relative ionization efficiency (predicted abundance of double (2+) and triply (3+) charged peptide ions of over 10% of total abundance).

Two additional protein virus subsets were created to demonstrate the effect of variant inclusion on peptide sample sets, restricting the analysis to only the most frequently observed and the gene translation products that undergo mutation, requiring 100% and 95% variant coverage respectively. The top three panels (FIG. 5A, FIG. 5C and FIG. 5E) represent the 100% variant detection coverage embodiment; whereas the bottom three panels (FIG. 5B, FIG. 5D and FIG. 5F) are the 95% embodiment. Further, panels shown in FIG. 5A and FIG. 5B illustrate cases where all protein variants are considered; FIG. 5C and FIG. 5D illustrate embodiments where only the high frequently occurring virus genes/proteins (HA (hemagglutinin), M (matrix protein 1 and matrix protein 2), NA (neuraminidase), NB (glycoprotein NB), NP (nucleoprotein), NS (non-structural protein 1), PA (polymerase acidic protein), PB1 (RNA-directed RNA polymerase catalytic subunit) and PB2 (polymerase basic protein 2)) are considered; and FIG. 5E and FIG. 5F illustrate the embodiment in which genes/proteins present are the ones that can undergo mutation, gene translation (HA (hemagglutinin) and NA (neuraminidase)). The difference between 100% coverage and 95% coverage has a large impact on the targeted number of peptides. For example, reviewing panels shown in FIG. 5A and FIG. 5B, the 100% coverage results in 108 peptides, whereas the 95% coverage is reduced to 61 peptides. Further by filtering the data regarding the variants, from all variants considered to only genes that undergo mutation (panel of FIG. 5A→FIG. 5C→FIG. 5E) a further reduction in the target list of peptides is achievable.

Example 3: Selection of Peptides for Detection of all Known Protein Virus Variants. In this example, the analysis of Example 2 is extended to including circulating influenza virus proteins based on WHO vaccine development recommendations (https://www.who.int/influenza/vaccines/virus/recommendations/202002_recommendation.pdf). The proteins and amino acid sequences from predicted circulating influenza viruses were obtained from the UniRef section (100% sequence identity clusters) and the reviewed entries of the UniProt Protein Knowledge Database.

In this particular example, demonstrating that tailoring of the pre-analysis step, based on, for example, expert domain knowledge, can be readily achieved, the filtering step prior to the MCMC analyses, included limiting the in silico generated peptides (digestion) to a subset of peptides with a sequence length of 5 to 20 amino acid, and allowing for one missed cleavage.

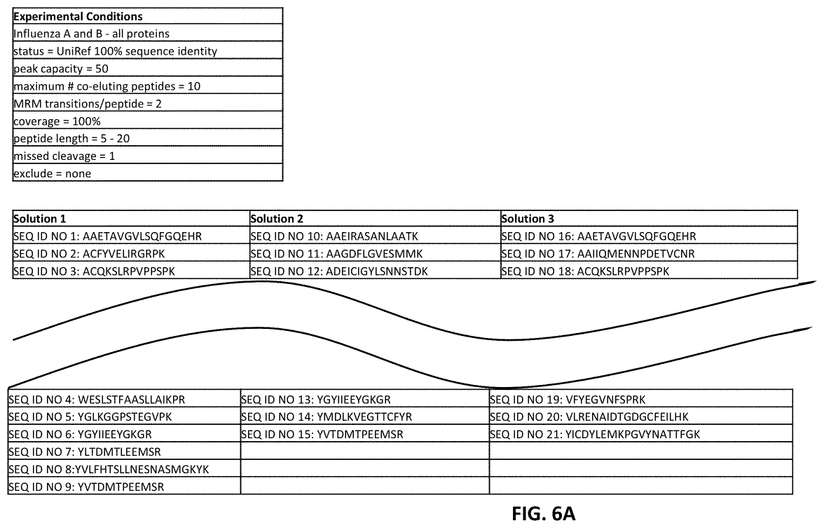

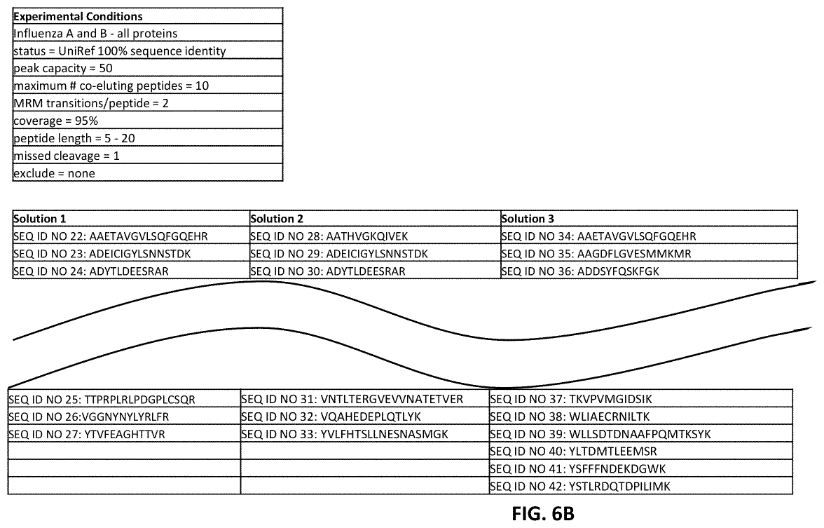

In silico determined physicochemical analyte properties included normalized retention time and relative ionization efficiency (predicted abundance of double (2+) and triply (3+) charged peptide ions of over 10% of total abundance). For the peptide selection of both circulating influenza (A and B) variants and circulating variants plus UniProt reviewed influenza (A and B variants 100% and 95% variant coverage were considered, representing four cases in total. For all these cases, three possible solutions were determined. For the 100% variant coverage, each possible solution (i.e., Solution 1, Solution 2, and Solution 3) included 146 or 149 sequences. And for the 95% variant coverage, each possible solution included 84 or 87 sequences. FIG. 6A provides the conditions and illustrates a portion of each of the three solutions for 100% variant coverage; whereas FIG. 6B provides the conditions and illustrates a portion of each of the three solutions for 95% variant coverage.

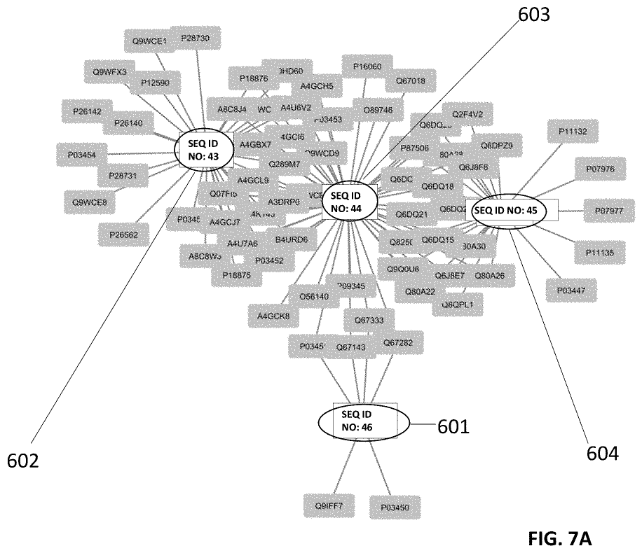

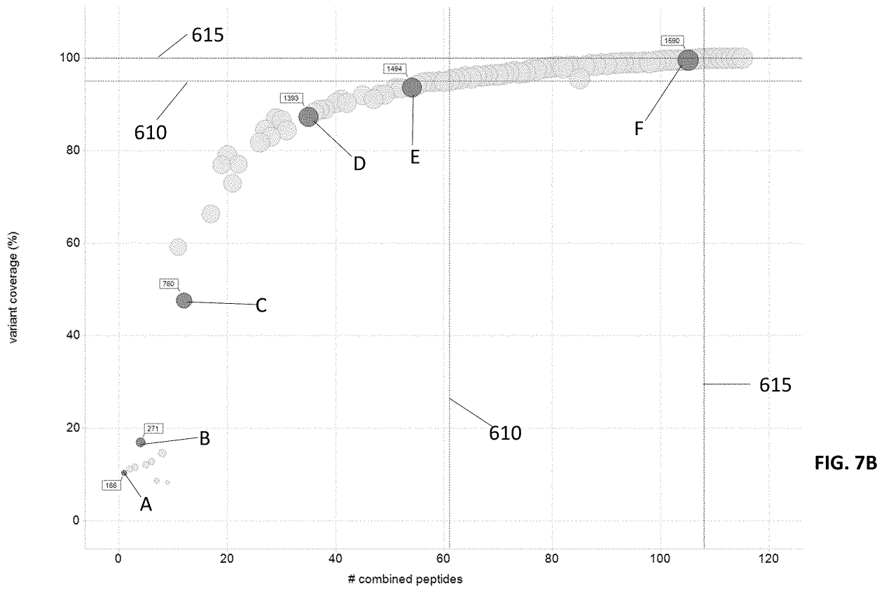

FIG. 7A illustrates an intermediate combination of peptides selected by the MCMC processing to obtain a given variant coverage. That is, FIG. 7A is a schematic representation of a possible intermediate combination of peptides selected to achieve a given final detection coverage for all UniProt Protein Knowledge Database influenza virus proteins with the central nodes (601, 602, 603, and 604) representing high frequency peptides (mapping to the highest number of virus protein variants, while also meeting all other selected criteria, i.e., a PC value of 50, not exceeding the maximum of ten co-eluting peptides, two MRM transitions, and charge state and abundance criteria). In the possible (but not only) solution illustrated in FIG. 7A, a combination of 4 peptides (601, 602, 603, and 604) cover (or map to) 112 proteins, providing 7% variant coverage. Different combinations and sets of combinations are feasible to achieve any desired variant coverage.

FIG. 7B is an example of the magnitude of the coverage that can be achieved by selecting an appropriate combined number of peptides for all UniProt Protein Knowledge Database influenza viruses. Lines 610 are used to illustrate the minimum number of peptides that have to be combined to achieve 95% detection coverage, whereas lines 615 are used to illustrate the minimum number of peptides that have to be combined to achieve 100% detection coverage for all UniProt Protein Knowledge Database influenza viruses. As FIG. 6B is generated from data includes for all variants of influenza A and B combined (i.e., for all UniProt Protein Knowledge Database influenza viruses), the graphical representations align to the virtual chromatograms shown in panels FIG. 5A and FIG. 5B. That is, FIG. 5A provides 100% coverage data and indicates that a combination of 108 peptides are to be selected. Lines 615 of FIG. 7B also represent 100% coverage and also reflect 108 combined peptides. Similarly, lines 610, which represent 95% coverage indicate a combination of 61 peptides. While, panel shown in FIG. 5B indicates the combination of 61 peptides.

Additional information reflected in the graph of FIG. 7B includes the number of proteins which map or are covered by the #combined peptides. In FIG. 6A, a total of 112 proteins are covered by the combination of 4 peptides (601, 602, 602, and 604). FIG. 7B, provides six additional data examples regarding the number of proteins that will be covered by the associated number of combined peptides. Points A, B, C, D, E, and E shown in FIG. 7B are each associated with a #combined peptides which can be determined from the x-axis of FIG. 7B, the size of the markers represents the associated number of proteins. The exemplary data points shown in FIG. 7B are as follows: point A, 1 peptide covering 166 proteins; point B, 4 peptides covering 271 proteins; point C, 12 peptides covering 760 proteins; point D, 35 peptides covering 1383 proteins; point E, 54 peptides covering 1494 proteins; and point F, 105 peptides covering 1590 proteins. Using the information in FIG. 7B, one can determine or leverage variant coverage demands with the #combined peptides complexities to determine an appropriate solution.

The above examples illustrate the effects filtering criteria and statistical analysis approaches to derive a scoring function imparts on number of selection results. This approach can be used to analyze the simulation of an enormous amount of laboratory experiments to achieve actual workflow peptides for the desired analysis.

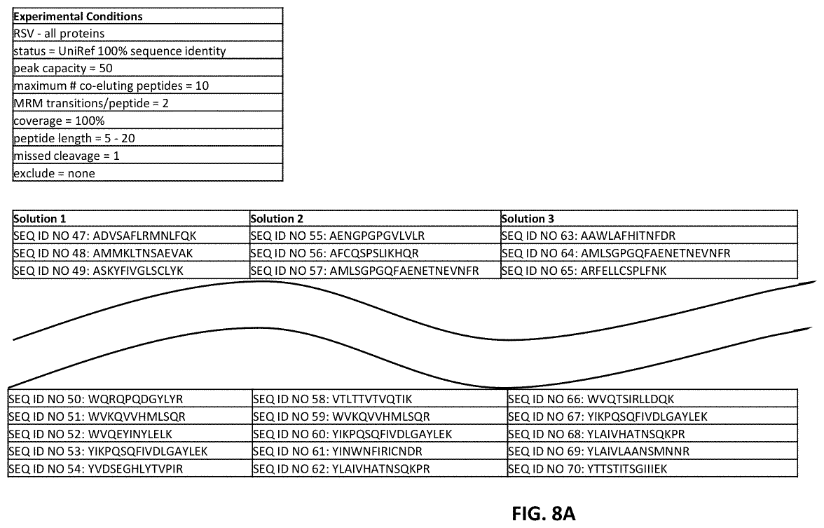

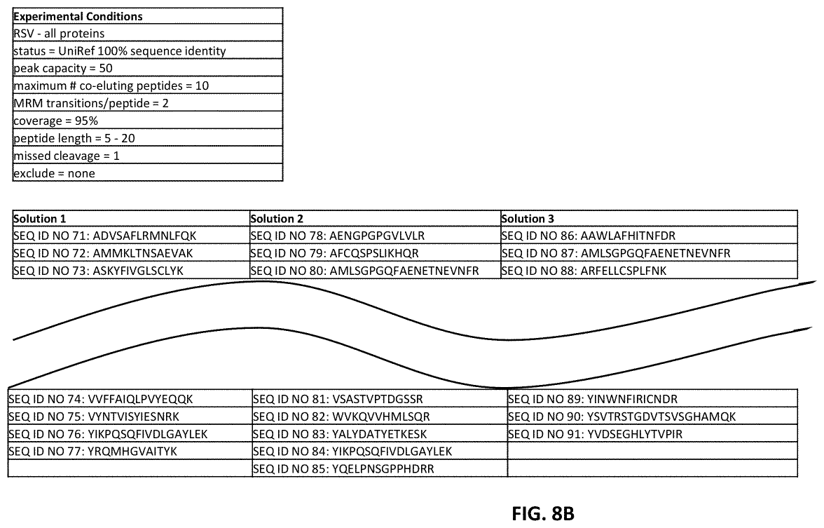

Example 4: Selection of Peptides for Detection of all Known Protein Virus—Respirator Syncytial Virus (RSV). As another example, solutions for RSV with the proteins obtained from UniRef section (100% sequence identity clusters) and reviewed entries of the UniProt Protein Knowledge Database. The pre-analysis filtering steps were identical to Example 3 and 95% and 100% variant coverage solutions determined. Three possible solutions for both cases were determined. For the 100% variant coverage, each possible solution (i.e., Solution 1, Solution 2, and Solution 3) included 62 sequences. And for the 95% variant coverage, each possible solution included 53 or 54 sequences. FIG. 8A provides the conditions and illustrates a portion of each of the three solutions for 100% variant coverage; whereas FIG. 8B provides the conditions and illustrates a portion of each of the three solutions for 100% variant coverage.

Table 1 below summarizes Examples 1˜4 and identifies the optional protein subsets.

| | | | | | | Excluded | | |
|---|---|---|---|---|---|---|---|---|
| | | | | | | amino | | |
| | | | | | # | acid | | Protein^{†} |
| | | | Knowledge | | amino | containing | Missed | subset |
| Ex | case | virus | db | Conditions | acids | peptides | cleavages | (optional) |
| 1 | differentiation | SARS-CoV- | UniProt | Peak capacity = | 5-15 | M | 0 | — (all) |
| | | 2 | (reviewed) | 50 | | | | |
| | | SARS-CoV | | Max co- | | | | |
| | | MERS-CoV | | eluting | | | | |
| | | Cov 229E | | peptides = 10 | | | | |
| | | CoV OC43 | | MRM | | | | |
| | | CoV NL63 | | transitions/ | | | | |
| | | CoVHKU1 | | peptide = 2 | | | | |
| 2 | coverage | Influenza | UniProt | Peak capacity = | 5-15 | M | 0 | — (all) |
| | | A | (reviewed) | 50 | | | | |
| | | Influenza | | Max co- | | | | |
| | | B | | eluting | | | | |
| | | | | peptides = 10 | | | | |
| | | | | MRM | | | | |
| | | | | transitions/ | | | | |
| | | | | peptide = 2 | | | | |
| 3 | coverage | Influenza | UniRef | Peak capacity = | 5-15 | M | 0 | NP |
| | | A | 100% | 50 | | | | HA |
| | | Influenza | sequence | Max co- | | | | NA |
| | | B | identity | eluting | | | | NS |
| | | | | peptides = 10 | | | | M |
| | | | | MRM | | | | PB1 |
| | | | | transitions/ | | | | PB2 |
| | | | | peptide = 2 | | | | NB |
| 4 | coverage | Respirator | UniRef | Peak capacity = | 5-15 | M | 0 | — (all) |
| | | Syncytial | 100% | 50 | | | | |
| | | Virus | sequence | Max co- | | | | |
| | | (RSV) | identity | eluting | | | | |
| | | | | peptides = 10 | | | | |
| | | | | MRM | | | | |
| | | | | transitions/ | | | | |
| | | | | peptide = 2 | | | | |

^{†}abbreviations (nucleocapsid (N) and nucleoprotein (NP) are interchangeably used in literature and protein knowledge databases):
— Influenza A/B: haemagglutinin (HA); matrix protein (M); nucleoprotein (NP); non-structural protein 1 (NS), matrix protein 1 and matrix protein 2 (M), RNA-directed RNA polymerase catalytic subunit (PB1), polymerase basic protein 2 (PB2), glycoprotein NB (NB)

In additional embodiments, further separation or filtering steps may be employed in the analysis, including, but not limited to ion mobility separation. These separations may be modelled as part of an in silico experimental design workflow. In the case of ion mobility separation, arrival times of peptides or peptide fragments at a particular point in the instrument (for example a mass filter) may be determined using calibration information and/or values from previous experiments or literature.

What is claimed is:

1. A method for selecting a combination of peptides to identify one or more disease states in a subject using mass spectrometry, the method comprising:
selecting the one or more disease states;
collecting information on proteins associated with the one or more disease states being present within the subject;
in silico digesting individual proteins associated with the one or more disease state to obtain possible workflow peptides;
collecting filtering data associated with the possible workflow peptides;
analyzing the possible workflow peptides for coverage of the one or more disease states and for mass spectrometry detection and resolution; and
selecting the combination of peptides from the possible workflow peptides based on the analyzing step.

2. The method of claim 1, wherein two or more disease states are selected.

3. The method of claim 2, wherein three disease states are selected.

4. The method of claim 1, wherein two or more variants of the one or more disease states are selected.

5. The method of claim 1, wherein the filtering data comprises physicochemical data.

6. The method of claim 1, wherein the filtering data comprises homology data.

7. The method of claim 1, wherein the filtering data comprises abundance of peptide data (frequency %).

8. The method of claim 1, wherein the filtering data comprises data associated with the one or more disease states.

9. The method of claim 1, wherein the filtering data comprises physicochemical data and abundance of peptide data.

10. The method of claim 5, wherein the physicochemical data comprises ionization efficiency and/or retention time.

11. The method of claim 5, wherein the physicochemical data comprises one or more of (a) length of possible workflow peptides; (b) MRM transition data on co-eluting or close eluting possible workflow peptides; and (c) amino acid sequences contained within the possible workflow peptides.

12. The method of claim 1, wherein the filtering data comprises data on methionine being present within amino acid sequences contained within the possible workflow peptides.

13. The method of claim 1, further comprising eliminating possible workflow peptides using the filtering data prior to analyzing the possible workflow peptides for coverage of the one or more disease states and for mass spectrometry detection and resolution.

14. The method of claim 1, further comprising eliminating possible workflow peptides after analyzing the possible workflow peptides for coverage of the one or more disease states and for mass spectrometry detection and resolution using filtering data.

15. The method of claim 1, wherein analyzing the possible workflow peptides for coverage of the one or more disease states and for mass spectrometry detection and resolution comprises applying a statistical approach.

16. The method of claim 15, wherein the statistical approach comprises Bayesian inference.

17. The method of claim 15, wherein the statistical approach comprises a Markov Chain Monte Carlo algorithm.

18. The method of claim 1, wherein analyzing the possible workflow peptides for coverage of the one or more disease states and for mass spectrometry detection and resolution comprises analyzing coverage for a yes/no result for the disease state.

19. The method of claim 1, wherein analyzing the possible workflow peptides for coverage of the one or more disease states and for mass spectrometry detection and resolution comprises analyzing coverage for a determination of a particular variant of the disease state.

20. The method of claim 1, wherein analyzing the possible workflow peptides for coverage of the one or more disease states and for mass spectrometry detection and resolution comprises analyzing coverage for a determination of a particular disease state from a group of possible disease states.
